(19) United States
(12) Patent Application Publication
(10) Pub. No.: US 2022/0218185 A1
(43) Pub. Date: Jul. 14, 2022

(54) APPARATUS AND METHOD FOR GUIDING INSPECTION OF LARGE INTESTINE BY USING ENDOSCOPE

(71) Applicant: Jihwan KO, Busan (KR)

(72) Inventor: Jihwan KO, Busan (KR)

(21) Appl. No.: 17/190,518

(22) Filed: Mar. 3, 2021

Related U.S. Application Data
(63) Continuation of application No. PCT/KR2021/000929, filed on Jan. 22, 2021.

(30) Foreign Application Priority Data
Jan. 14, 2021 (KR) 10-2021-0005413

(51) Int. Cl. A61B 1/01; A61B 1/00; A61B 1/06; G06N 20/00; A61B 90/00

(52) U.S. Cl. CPC A61B 1/01; A61B 1/00045; A61B 90/39; A61B 1/00009; G06N 20/00; A61B 1/0661

(57) ABSTRACT

The inventive concept provides a method for guiding an inspection of a large intestine by using an endoscope, being performed by an apparatus, including receiving an image captured by the endoscope introduced into a large intestine of a patient, in real time, recognizing each of section images containing at least one wrinkle in the large intestine, in the image, displaying a first visual effect of representing the wrinkle in each of the section images, determining whether a rear surface of the wrinkle in each of the section images is photographed, and displaying a second visual effect of representing, in at least one of the section images, in which a rear surface of a wrinkle has not been photographed, that the rear surface of the wrinkle in the at least one of the section images has not been photographed.

The present application is a continuation of International Patent Application No. PCT/KR2021/000929, filed on Jan. 22, 2021, which is based upon and claims the benefit of priority to Korean Patent Application No. 10-2021-0005413 filed on Jan. 14, 2021. The disclosures of the above-listed applications are hereby incorporated by reference herein in their entirety.

### BACKGROUND

Embodiments of the inventive concept relate to guide of an inspection of a large intestine, and more particularly, relate to an apparatus and a method for guiding an inspection of a large intestine by using an endoscope.

In general, an endoscope inspection of a large intestine is adapt to basically prevent a large intestine cancer by discovering a polyps that is a portion of a tissue that rises on an inner wall of a large intestine, which is a prodromal change of the large intestine cancer, in a small size and removing the polyps. In detail, an interval cancer may be prevented by discovering a polyps through a large intestine endoscope and removing the polyps. However, it is known that a polyps cannot be discovered in up to about 30% of the large intestine endoscope inspections.

This is because finding a large intestine polyps hidden on a rear surface of a wrinkle, among the wrinkles in the large intestine, has to depend on a concentration force of a doctor who inspects the large intestine in reality, and method for evaluating the inspection only includes an indirect method, for example, of determining whether a time for the entire inspection is 6 minutes or more. Even a doctor who makes a thorough observation may observe the already observed sites again or may not observe sites that have not been observed according to his or her inspection style as if persons find treasures in a treasure hunt in different ways.

Accordingly, a method for identifying a plurality of wrinkles in a large intestine of a patient through an endoscope without omitting the wrinkles when performing a large intestine endoscope is necessary.

### SUMMARY

An aspect of the inventive concept for solving the above-mentioned problems is to recognize a wrinkle of a large intestine in an endoscope image of a large intestine and provide the recognized wrinkle to an expert who operates the large intestine endoscope when the large intestine endoscope is operated.

In detail, the aspect of the inventive concept is to display the wrinkle of the large intestine recognized in the endoscope image of the large intestine while making the size of a visual effect different for the size of the wrinkle.

Furthermore, an aspect of the inventive concept is to provide a visual effect to a corresponding wrinkle when a rear surface of a large intestine wrinkle is not photographed through an endoscope, and remove the visual effect for the corresponding wrinkle when an expert photographs the rear surface of the corresponding wrinkle by using an endoscope.

The technical objects of the inventive concept are not limited to the above-mentioned ones, and the other unmentioned technical objects will become apparent to those skilled in the art from the following description.

According to an embodiment, a method for guiding an inspection of a large intestine by using an endoscope, being performed by an apparatus, may include receiving an image captured by the endoscope introduced into a large intestine of a patient, in real time, recognizing each of section images containing at least one wrinkle in the large intestine, in the image, displaying a first visual effect of representing the wrinkle in each of the section images, determining whether a rear surface of the wrinkle in each of the section images is photographed, and displaying a second visual effect of representing, in at least one of the section images, in which a rear surface of a wrinkle has not been photographed, that the rear surface of the wrinkle in the at least one of the section images has not been photographed.

Here, the recognition may be performed based on light irradiated into the large intestine by the endoscope.

Furthermore, the recognition may include recognizing each of the section images through a deep learning model, and the deep learning model may be a model that is machine-learned based on wrinkle data in images of the large intestines of a plurality of patients, which are obtained from external annotators, and change amounts of shades due to light irradiated in the large intestines, and blood vessels.

Here, the first visual effect may include a visual effect displayed by each of the markers on the corresponding wrinkle in each of the section images, and the size of each of the markers may be determined based on the size of the corresponding wrinkle.

Furthermore, the method may further include, when the rear surface of the corresponding wrinkle is photographed, deleting the second visual effect.

In addition, another method for realizing the inventive concept, another system, and a computer readable recording medium for recording a computer program for executing the method may be further provided.

### BRIEF DESCRIPTION OF THE FIGURES

The above and other objects and features will become apparent from the following description with reference to the following figures, wherein like reference numerals refer to like parts throughout the various figures unless otherwise specified, and wherein:

FIG. 1 is a block diagram schematically illustrating an apparatus for providing a large intestine inspection guide using an endoscope according to the inventive concept;

FIG. 2 is a block diagram schematically illustrating that a deep learning model used for a large intestine inspection guide is learned according to the inventive concept;

FIG. 3 is an exemplary view illustrating a process of providing a large intestine inspection guide using an endoscope by a processor of an apparatus according to the inventive concept;

FIGS. 4A to 4D are exemplary views illustrating that a first visual effect of representing a wrinkle on an image captured by an endoscope introduced into a large intestine of a patient is displayed according to the inventive concept;

FIGS. 5A to 5D are exemplary views illustrating that a second visual effect representing a wrinkle, in which an image of a rear surface of a wrinkle, on which a first visual effect is displayed, is not captured, is displayed according to the inventive concept; and

FIG. 6 is a flowchart illustrating a process of guiding a large intestine inspection by using an endoscope by a processor of an apparatus according to the inventive concept.

### DETAILED DESCRIPTION

The above and other aspects, features, and advantages of the inventive concept will become apparent from the following description of the following embodiments given in conjunction with the accompanying drawings. However, the inventive concept is not limited by the embodiments disclosed herein but will be realized in various different forms, and the embodiments are provided only to make the disclosure of the inventive concept complete and fully inform the scope of the inventive concept to an ordinary person in the art, to which the inventive concept pertains, and the inventive concept will be defined by the scope of the claims.

The terms used herein are provided to describe the embodiments but not to limit the inventive concept. In the specification, the singular forms include plural forms unless particularly mentioned. The terms “comprises” and/or “comprising” used herein does not exclude presence or addition of one or more other elements, in addition to the aforementioned elements. Throughout the specification, the same reference numerals denote the same elements, and “and/or” includes the respective elements and all combinations of the elements. Although “first”, “second” and the like are used to describe various elements, the elements are not limited by the terms. The terms are used simply to distinguish one element from other elements. Accordingly, it is apparent that a first element mentioned in the following may be a second element without departing from the spirit of the inventive concept.

Unless otherwise defined, all terms (including technical and scientific terms) used herein have the same meaning as commonly understood by those skilled in the art to which the inventive concept pertains. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein.

Hereinafter, exemplary embodiments of the inventive concept will be described in detail with reference to the accompanying drawings.

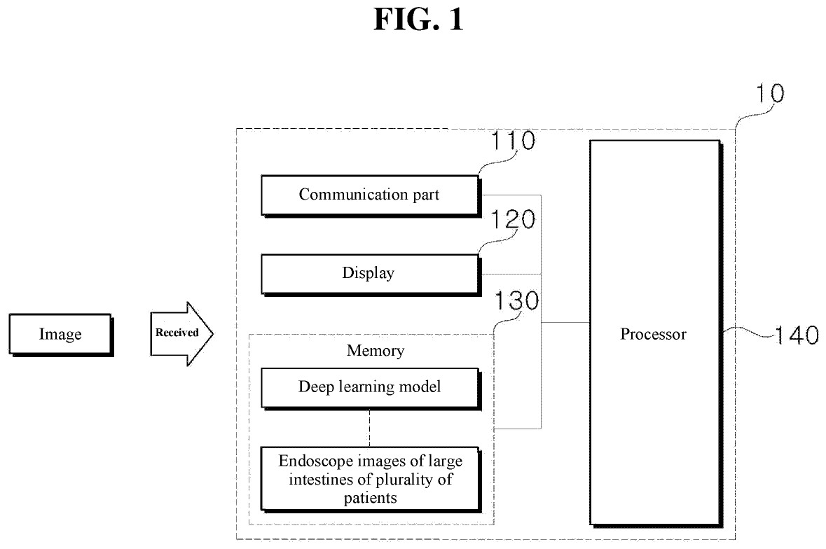

FIG. 1 is a block diagram schematically illustrating an apparatus 10 for providing a large intestine inspection guide using an endoscope according to the inventive concept.

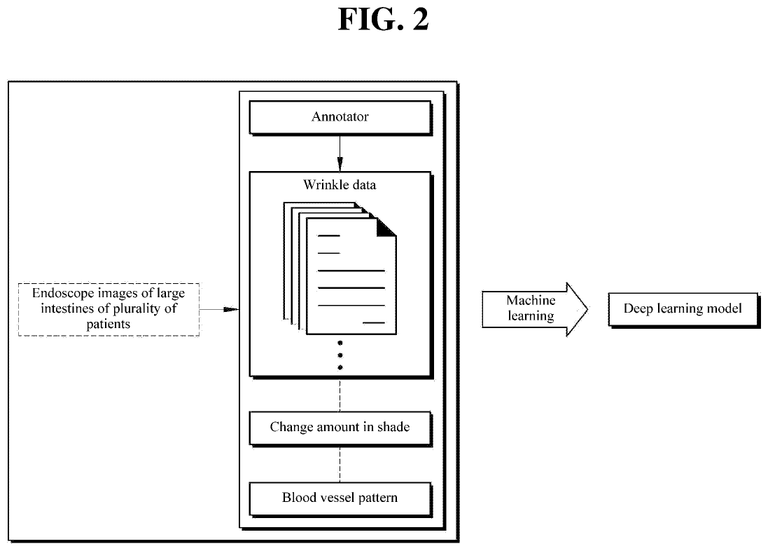

FIG. 2 is a block diagram schematically illustrating that a deep learning model used for a large intestine inspection guide is learned according to the inventive concept.

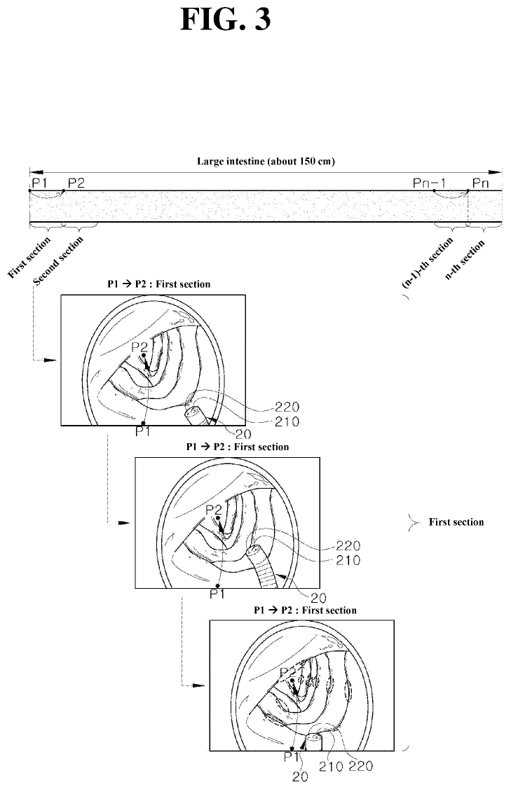

FIG. 3 is an exemplary view illustrating a process of providing a large intestine inspection guide using an endoscope by a processor 140 of an apparatus 10 according to the inventive concept.

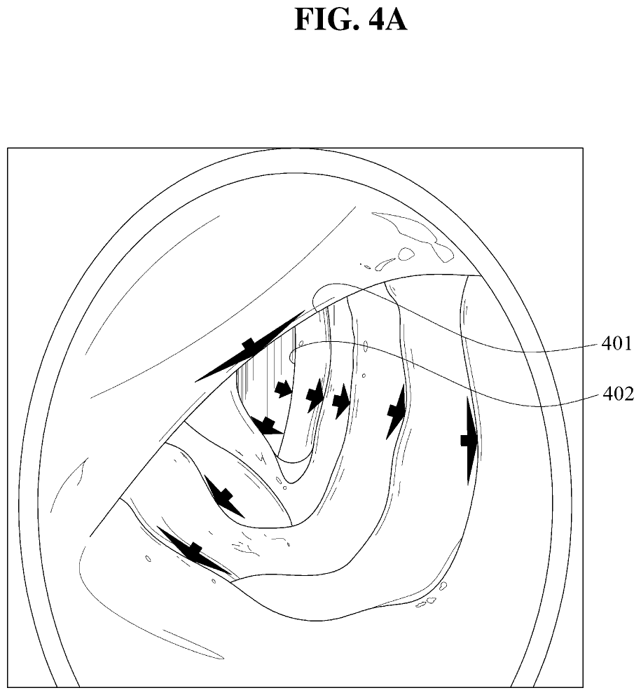

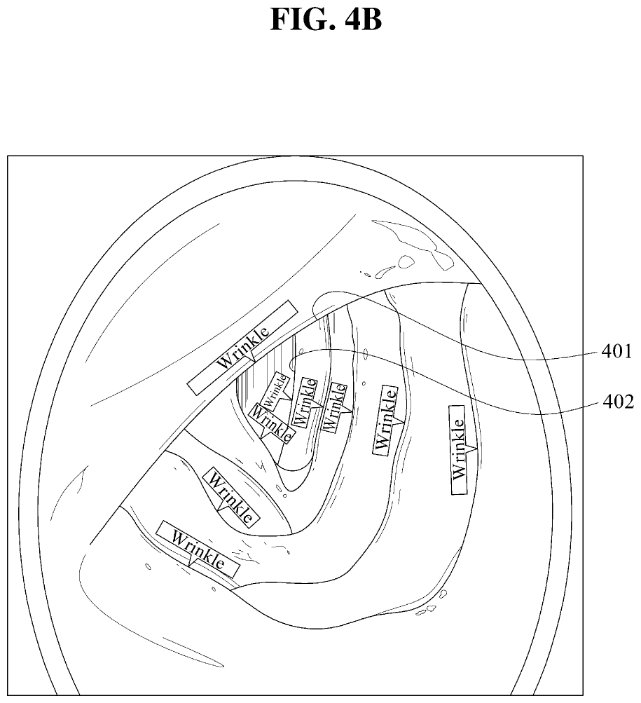

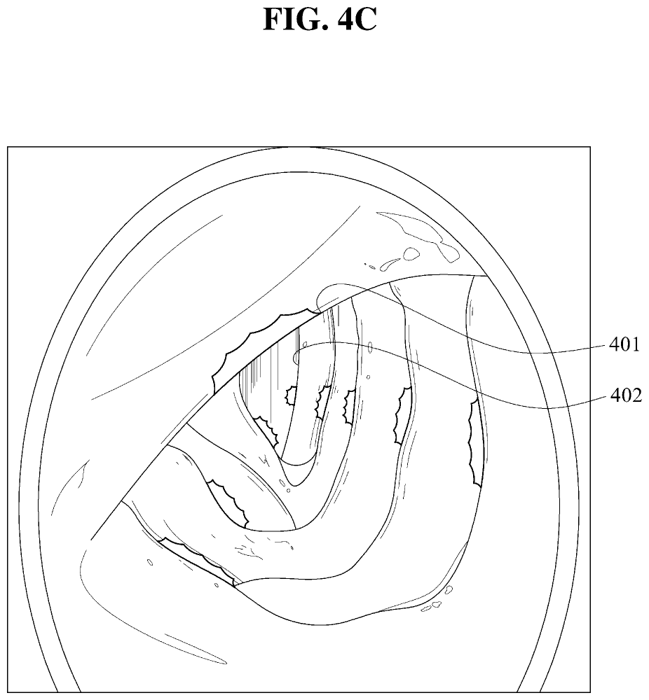

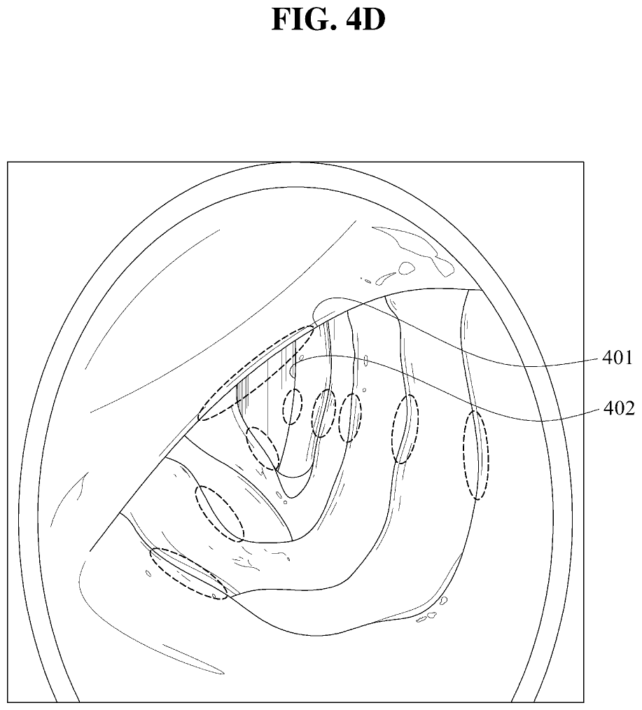

FIGS. 4A to 4D are exemplary views illustrating that a first visual effect that represents a wrinkle on an image captured by an endoscope introduced into a large intestine of a patient is displayed according to the inventive concept.

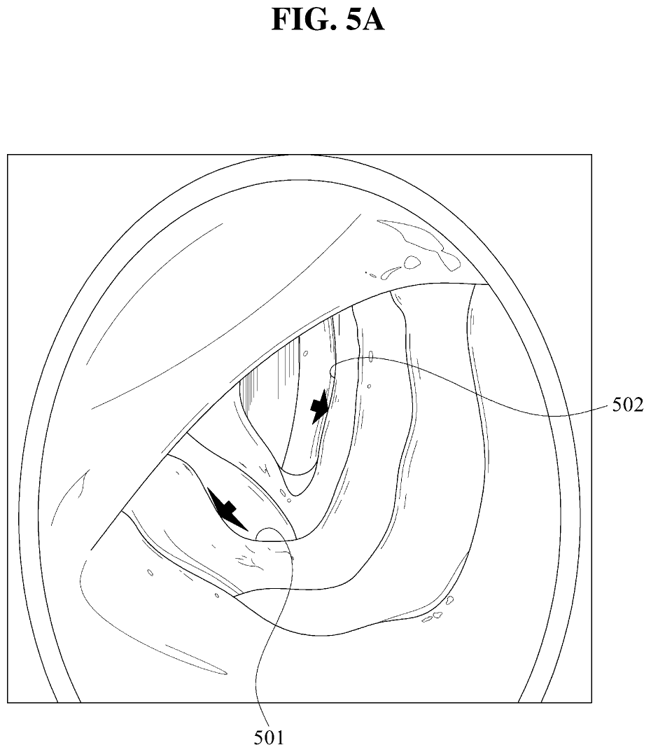

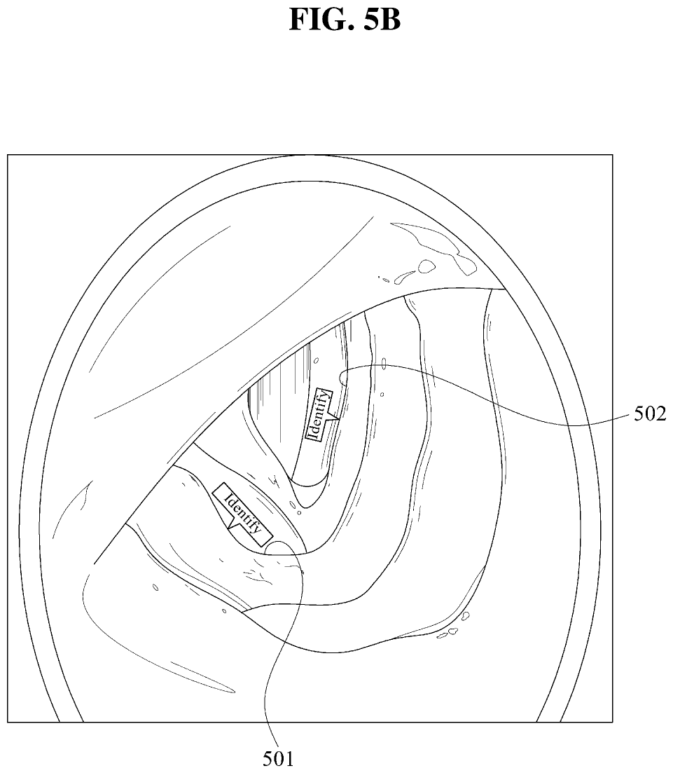

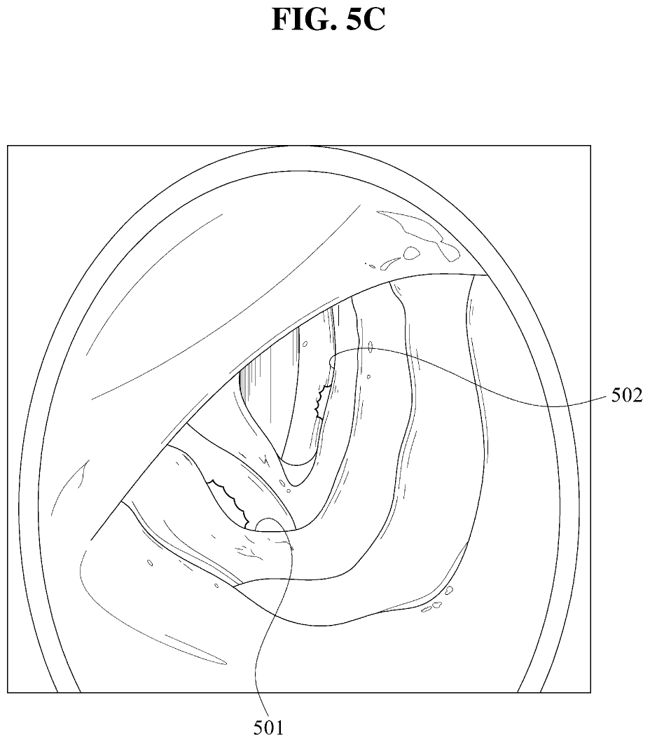

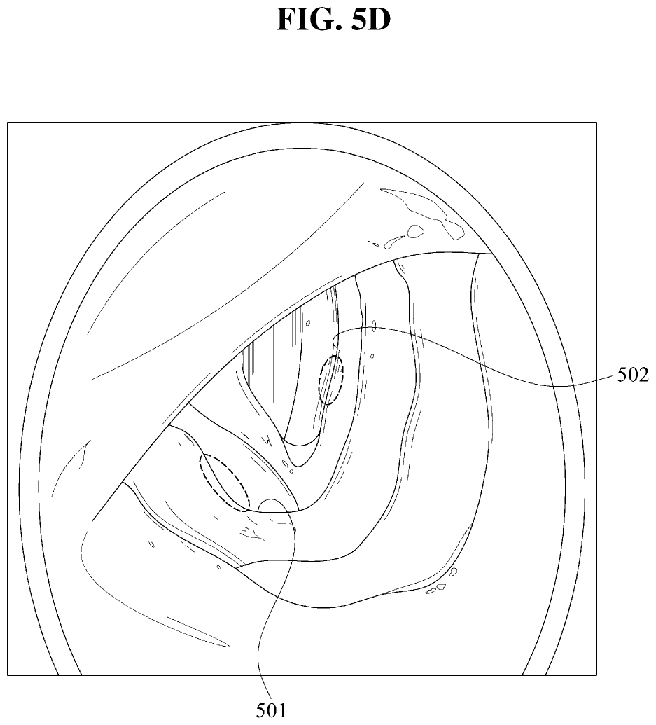

FIGS. 5A to 5D are exemplary views illustrating that a second visual effect representing a wrinkle, in which an image of a rear surface of a wrinkle, on which a first visual effect is displayed, is not captured, is displayed according to the inventive concept.

Hereinafter, an apparatus 10 for providing a large intestine inspection guide using an endoscope according to the inventive concept will be described with reference to FIGS. 1 to 5D. Here, the apparatus 10 may be realized as a server device as well as a local computer device.

The apparatus 10 may have an effect of easily discovering a polyps that may be located behind a wrinkle of a large intestine by recognizing the wrinkle of the large intestine in an endoscope image of the large intestine and providing the endoscope image of the large intestine to an expert who operates the large intestine endoscope when a large intestine endoscope is performed.

In detail, the apparatus 10 may have an effect of causing a wrinkle to be identified as a whole while only a portion of the wrinkle is identified when the size of the wrinkle is large, by displaying the large intestine recognized in an endoscope image of a large intestine such that visual effects are different according to the size of the wrinkle of the large intestine.

Furthermore, the apparatus 10 may have an effect of clearly determining a site, at which a rear surface of a wrinkle has been identified, and a site, at which a rear surface of a wrinkle has not been identified, by providing a visual effect for a corresponding wrinkle of a large intestine when the rear surface of the wrinkle has not been photographed through an endoscope 20, and removing a visual effect for the corresponding wrinkle when an expert has photographed the rear surface of the corresponding wrinkle by using the endoscope 20. Here, the endoscope 20 is a device that is inserted into a large intestine to observe biological tissues in the large intestine, and may include a camera 210, a lighting 220, and the like.

First, referring to FIG. 1, the apparatus 10 includes a communication unit 110, a display 120, a memory 130, and the processor 140. Here, the apparatus 10 may include components, the number of which is smaller than or larger than the number of the components illustrated in FIG. 1.

The communication unit 110 may include one or more modules that allow wireless communication between the apparatus 10 and a wireless communication system, between the apparatus 10 and the endoscope 20, and between the apparatus 10 and an external device (not illustrated). Furthermore, the communication unit 110 may include one or more modules that connect the apparatus 10 to one or more networks.

The communication unit 110 may receive an image captured by the endoscope introduced into the large intestine of the patient in real time. Furthermore, the communication unit 110 may receive images captured by endoscopes introduced into large intestines of a plurality of patients, or may receive images captured by an endoscope introduced into a large intestine of a patient several times.

Furthermore, the communication unit 110 may receive wrinkle data obtained by a medical staff as an example of an annotator, for an image of a large intestine of a patient captured through an endoscope for learning by a deep learning model.

The display 120 may realize a touchscreen by forming a mutual layer structure with a touch sensor or integrally forming the touchscreen with the touch sensor. The touchscreen may provide an input interface between the apparatus 10 and a user and provide an output interface between the apparatus 10 and the user at the same time.

The display 120 may display various pieces of information generated by the processor 140 to the user to provide the information to the user and receive various pieces of information at the same time.

In more detail, the display 120 may display a first visual effect of representing a wrinkle for each of sections of an image. Furthermore, the display 120 may display a second visual effect of representing, in at least one of the section images, in which a rear surface of a wrinkle has not been photographed, that a rear surface of a wrinkle in the at least one of the section images has not been photographed.

The memory 130 may store information that supports various functions of the apparatus 10. The memory 130 may store a plurality of application programs driven by the apparatus 10, and data and instructions for an operation of the apparatus 10. At least some of the application programs may be loaded down from an external server (not illustrated) through wireless communication. Furthermore, at least some of the application programs may be present for the basic functions of the apparatus 10. Meanwhile, the application programs may be stored in the memory 130 and be installed on the apparatus 10 to be driven to allow the processor 140 to perform operations (or functions) of the apparatus 10.

The memory 130 may store a deep learning model for recognizing a wrinkle in the image captured by the endoscope introduced into the large intestine of the patient. Here, the deep learning model may include a convolutional neural network (hereinafter, referred to as a CNN), but, without being limited thereto, may be formed of neural network of various structures.

The CNN may be structured to repeat several times a convolution layer that creates a feature map by applying a plurality of filters for each of the areas of the image and a pooling layer that allows a feature that does not vary according to a change in location or rotation to be extracted, by spatially integrating the feature maps. Through this, features of various levels, including features of a low level such as points, lines, and planes to complex and meaningful features of a high level, may be extracted.

The convolution layer may take a nonlinear activation function in a product of a filter and a local receptive field, for respective patches of the input image to obtain a feature map. In comparison with another network structure, the CNN may have a feature of using a filter having a sparse connectivity and shared weights. The connection structure may reduce the number of seed trees, which are to be learned, and may improve prediction performance as a result by efficiently learning through an inverse-propagation algorithm.

The pooling layer or the sub-sampling layer may generate a new feature map by utilizing local area information of a feature map obtained from the previous convolution layer. In general, a feature map newly created by a pooling layer is reduced to a size that is smaller than the original feature map, and representative pooling method may include max pooling of selecting a maximum value of a corresponding area in a feature map and average pooling of obtaining an average value of a corresponding area in a feature map. A feature map of a pooling layer generally may be less influenced by the location of an arbitrary structure or pattern that is present in an input image than a feature map of the previous layer. That is, the pooling layer may extract a feature that is more robust to a local change, such as noise or distortion, in an input image or a previous feature map, and the feature may take an important role in classification performance. Furthermore, another role of a pooling layer is to reflect a feature of a wider area as it goes toward an upper learning layer in a deep structure, and create a feature of reflecting a location feature in a lower layer and reflecting a feature of a more abstract whole image in an upper layer while the feature extraction layers are stacked.

In this way, the feature finally extracted through repetition of a convolution layer and a pooling layer may be used for learning and predicting a classification model as a classification model, such as a multi-layer perception (MLP) or a support vector machine (SVM), is coupled in the form of a fully-connected layer.

The memory 130 may store the image obtained through the communication unit 110. Furthermore, the memory 130 may store images captured by endoscopes introduced into large intestines of a plurality of patients, or images captured by an endoscope introduced into a large intestine of a patient several times.

Furthermore, the memory 130 may store wrinkle data obtained by a medical staff as an example of an annotator, for an image of a large intestine of a patient captured through an endoscope for learning by a deep learning model.

The processor 140 may generally control an overall operation of the apparatus 10, in addition to an operation related to the application programs. The processor 140 may process signals, data, information, and the like that are input or output through the components discussed above, or may provide suitable information or functions to the user or process them by driving the application programs stored in the memory 130.

The processor 140 may control at least some of the components discussed with reference to FIG. 1 to drive the application programs stored in the memory 130. Moreover, the processor 140 may combine two or more of the components included in the apparatus 10 and operate them to drive the application programs.

Hereinafter, operations of the processor 140 will be described below in detail with reference to FIGS. 2 to 5.

The processor 140 may recognize each of section images, in which at least one wrinkle of a large intestine of a patient is included, in an image captured by the endoscope introduced into the large intestine, based on a deep learning model. That is, the processor 140 may recognize the at least one wrinkle in each of the section images, and may identify the size of the wrinkle.

Here, referring first to FIG. 2, the processor 140 may recognize each of the section images through the deep learning model, and the deep learning model may be a model that is machine-learned based on wrinkle data in large intestine images of a plurality of patients obtained from external annotators, a change amount of shade due to irradiated light in the large intestine, and a blood vessel pattern.

In detail, the processor 140 may obtain at least one image that is captured by the endoscopes introduced into the large intestines of the plurality of patients one or more times, and may obtain wrinkle data for the plurality of images, from the annotators. Here, the annotators may be experts who may identify the wrinkles of the large intestines well, and the plurality of images may be images for the endoscope applied to one patient several times or images for the endoscopes applied to a plurality of patients.

Thereafter, the processor 140 may machine-learn the first model based on the change amount of shade in the large intestine, the blood vessel, and the wrinkle data.

Here, the change amount of the shade may be obtained by determining, by the processor 140, whether the shade generated according to the light irradiated from the lighting 220 of the endoscope 20 to the wrinkle is changed by a preset threshold value or more. In detail, the processor 140 may determine that there is a wrinkle when the shade is changed darker by the preset threshold value or more.

Furthermore, the processor 140 may recognize a blood vessel pattern formed by the blood vessel in the endoscope image of the large intestine, and may recognize an area having a wrinkle in the endoscope image of the large intestine based on the recognized blood vessel pattern.

In detail, based on the recognized blood vessel pattern, the processor 140 may recognize a wrinkle area in the endoscope image of the large intestine by recognizing a portion that is viewed as if it is broken as a border line of the wrinkle when at least one blood vessel pattern in the endoscope image in the large intestine is viewed in the endoscope image of the large intestine as if the connection of the blood vessel pattern is broken according to the shade formed by the light from the lighting 220 of the endoscope 20, which is irradiated to the wrinkle.

Furthermore, the processor 140 may recognize the shape of the blood vessel in the endoscope image of the large intestine, and may recognize an area having a wrinkle in the endoscope image of the large intestine based on the recognized shape of the blood vessel.

In detail, based on the recognized shape of the blood vessel, the processor 140 may recognize a wrinkle area in the endoscope image of the large intestine by recognizing a portion, which is viewed as the shape of a bent hook, as the wrinkle, because the blood vessel is viewed in the endoscope image of the large intestine as if it is bent after passing through a bent portion of the wrinkle when the shape of the at least one blood vessel in the endoscope image of the large intestine is recognized as the shape of the hook.

Furthermore, the processor 140 may recognize the color of the at least one blood vessel in the endoscope image of the large intestine, and may recognize an area having a wrinkle in the endoscope image of the large intestine based on the recognized color of the blood vessel.

In detail, the processor 140 may recognize a wrinkle area in the endoscope image of the large intestine by recognizing a portion of the blood vessel, at which the change in the brightness of the blood vessel is a preset level or more, as a border line of the wrinkle because the brightness of the blood vessel changes when the blood vessel passes between the wrinkle and the rear surface of the wrinkle when the recognized change in the brightness of the blood vessel is the preset level or more.

The processor 140 may set a section from a point, at which the endoscope 20 is introduced, to a point identified according to light irradiated to the interior of the large intestine by the lighting 220 provided in the endoscope 20, as one section. That is, the processor 140 may divide the large intestine to “n” sections and recognize a wrinkle for each of the sections. Here, a large intestine of an adult is about 150 cm to 170 cm, and a distance to a point identified according to the light irradiated from the lighting 220 provided in the endoscope 20 may be about 10 cm to 15 cm. Accordingly, the processor 140 may divide the large intestine to about 10 to 15 sections, and may recognize a wrinkle for each of the sections.

In detail, the processor 140 may create a point, at which the endoscope 20 is introduced, and a point identified according to light irradiated to the interior of the large intestine by the lighting 220 provided in the endoscope 20, based on the deep learning model.

As an example, referring to FIG. 3, the processor 140 may set a section from a first point P1 of the image, which the endoscope 20 enters first, to a second point P2 identified according to the light irradiated to the interior of the large intestine by the lighting 220 of the endoscope 20, as a first section.

Furthermore, the processor 140 may set a section from the second point P2 to a third point P3 identified according to the light irradiated to the interior of the large intestine by the lighting 220 of the endoscope 20, as a second section, when the endoscope 20 is located at the second point P2 that is an ending point of the first section of the image.

Furthermore, the processor 140 may set a section from an (n−1)-th point P(n−1) to an n-th point Pn identified according to the light irradiated to the interior of the large intestine by the lighting 220 of the endoscope 20 and being a start point of the last section, at which the endoscope 20 may enter the interior of the large intestine, as an n-th section, when the endoscope 20 is located at the (n−1)-th point P(n−1) that is an ending point of the (n−1)-th section of the image.

Here, the second point P2 to an (n−1)-th point P(n−1), except for the first point P1 that is the first point, at which the endoscope 20 enters the large intestine and the n-th point Pn that is the start point of the final section, at which the endoscope 20 may enter the large intestine, may partially overlap the immediately previous section. Accordingly, the processor 140 may have an effect of inspecting the whole large intestine without any omitted section, by displaying the sections of the large intestine, which are to be identified through the endoscope 20, through the display 120, without omitting the sections.

Furthermore, the length of the first to n-th sections may be substantially the same or slightly different, and may be substantially the same because the intensities of the lights irradiated by the lighting 220 of the endoscope 20 are the same.

Referring to a section introduction frame A 301 of FIG. 3, the processor 140 may set a section from an introduction point of the image, which the endoscope 20 enters, to a final point that may be identified through light irradiated from the lighting 220 of the endoscope 20, as one section.

In more detail, the processor 140 may set a section from the first point P1 of the image, which the endoscope 20 enters first, to the second point P2 identified according to the light irradiated to the interior of the large intestine by the lighting 220 of the endoscope 20, as the first section.

Referring to a section middle frame 302 of FIG. 3, the processor 140 may display the first visual effect of representing the wrinkle in each of the section images. The first visual effect may include a visual effect of displaying a marker on the corresponding wrinkle in each of the section images. The size of each of the markers may be determined based on the size of the corresponding wrinkle.

Here, the processor 140 may recognize at least one wrinkle according to the above-described method in the introduction part of each of the section images and recognize a blood vessel pattern for each wrinkle to determine that, when there is the blood vessel pattern recognized through an image captured while the endoscope 20 passes through the introduction part of each of the sections, a wrinkle is at a location of the corresponding blood vessel pattern. Thereafter, the processor 140 may allow a medical expert to easily identify a wrinkle by displaying the first visual effect for the determined wrinkle.

In more detail, the processor 140 may display the first visual effect of representing the wrinkle in the first section. Here, the endoscope 20 may move into the interior of the large intestine while identifying the wrinkles of the large intestine one by one according to the first visual effect in the first section according to an operation of the medical expert.

As an example, referring to FIG. 4A, the processor 140 may display the first visual effect as a marker of an arrow shape for the size of each of the wrinkles of the first section. Accordingly, the size of the arrow shape of the first wrinkle 401, the size of which is large, may be larger than the size of the arrow shape of the second wrinkle 402, the size of which is small.

Next, referring to FIG. 4B, the processor 140 may display the first visual effect as a marker of a message shape for the size of each of the wrinkles of the first section. Accordingly, the size of the message shape of the first wrinkle 401, the size of which is large, may be larger than the size of the message shape of the second wrinkle 402, the size of which is small.

Next, referring to FIG. 4C, the processor 140 may display the first visual effect in the form of blinking for the size of each of the wrinkles of the first section. Accordingly, the size of the blinking of the first wrinkle 401, the size of which is large, may be larger than the size of the blinking of the second wrinkle 402, the size of which is small

Next, referring to FIG. 4D, the processor 140 may display the first visual effect as a circular marker for the size of each of the wrinkles of the first section. Accordingly, the size of the circular shape of the first wrinkle 401, the size of which is large, may be larger than the size of the circular shape of the second wrinkle 402, the size of which is small.

Here, the medical expert may easily recognize at least one wrinkle in each of the section images, based on the first visual effect displayed by the processor 140 through the display 120, may identify the rear surface of each of the wrinkles through the endoscope 20, and may photograph the rear surface.

Accordingly, the processor 140 may determine whether the rear surface of the wrinkle has been photographed for each of the section images.

Thereafter, referring to a section introduction frame B 303 of FIG. 3, the processor 140 may display a second visual effect of representing, in at least one of the section images, in which a rear surface of a wrinkle has not been photographed, that a rear surface of a wrinkle in the at least one of the section images has not been photographed. The second visual effect may include a visual effect of displaying a marker on the rear surface of the corresponding wrinkle in each of the section images. The size of each of the markers may be determined based on the size of the rear surface of the corresponding wrinkle.

Here, the endoscope 20 may move into the interior of the large intestine while identifying the wrinkles of the large intestine one by one according to the second visual effect in the first section. In detail, at least one wrinkle, the rear surface of which has not been photographed, may be identified for each of the areas according to the second visual effect, after the endoscope 20 recognizes the rear surface of each of the wrinkles for each of the areas and returns to a start point, from which the area is started.

First, referring to FIG. 5A, the processor 140 may display the second visual effect as a marker of an arrow shape for the size of each of the wrinkles of the first section, in which the rear surface of the wrinkle has not been photographed. Accordingly, the size of the arrow shape of the first wrinkle 501, the size of which is large, may be larger than the size of the arrow shape of the second wrinkle 502, the size of which is small.

Next, referring to FIG. 5B, the processor 140 may display the second visual effect as a marker of a message shape for the size of each of the wrinkles of the first section, in which the rear surface of the wrinkle has not been photographed. Accordingly, the size of the message shape of the first wrinkle 501, the size of which is large, may be larger than the size of the message shape of the second wrinkle 502, the size of which is small

Next, referring to FIG. 5C, the processor 140 may display the second visual effect in the form of blinking for the size of each of the wrinkles of the first section, in which the rear surface of the wrinkle has not been photographed.

Accordingly, the size of the blinking of the first wrinkle 501, the size of which is large, may be larger than the size of the blinking of the second wrinkle 502, the size of which is small.

Next, referring to FIG. 5D, the processor 140 may display the second visual effect as a circular marker for the size of each of the wrinkles of the first section, in which the rear surface of the wrinkle has not been photographed. Accordingly, the size of the circular shape of the first wrinkle 501, the size of which is large, may be larger than the size of the circular shape of the second wrinkle 502, the size of which is small

The processor 140 may delete the second visual effect when the rear surface of the corresponding wrinkle is photographed through the endoscope 20 based on the second visual effect. Accordingly, the processor 140 may certainly provide the first visual effect or the second visual effect to the wrinkle identified and not identified by the medical expert through the endoscope 20 to allow all the wrinkles to be identified without leaving any wrinkles that have not been identified, thereby increasing the precision of the inspection of the large intestine.

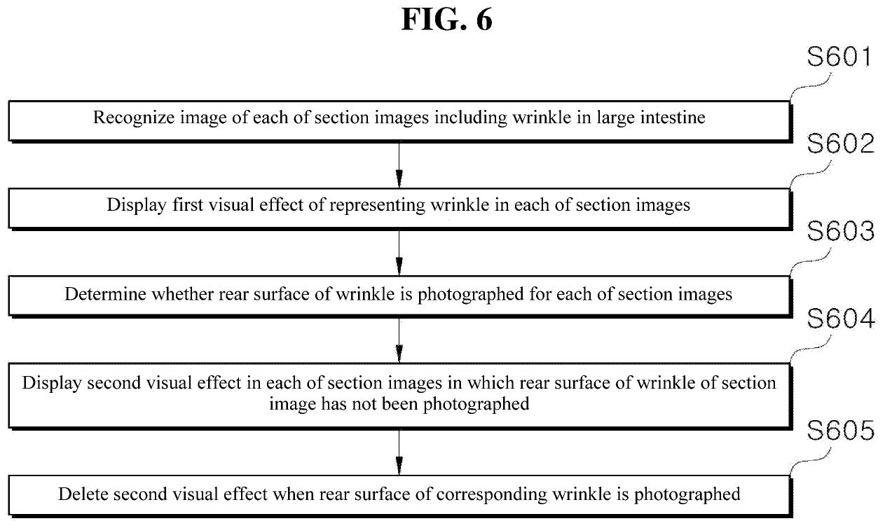

FIG. 6 is a flowchart illustrating a process of guiding a large intestine inspection using an endoscope by the processor 140 of the apparatus 10 according to the inventive concept. Here, an operation of the processor 140 may be performed by the apparatus 10.

First, the processor 140 may recognize each of the section images including at least one wrinkle in the large intestine in the image captured by the endoscope introduced into the large intestine of the patient, which has been received in real time through the communication unit 110 (S601).

Here, the processor 140 may recognize the wrinkle based on the light irradiated to the interior of the large intestine by the endoscope. Further, the processor 140 may recognize each of the section images through the deep learning model. Here, the deep learning model may be a model that is machine-learned based on wrinkle data in images of the large intestines of a plurality of patients, which are obtained from external annotators, and change amounts of shades due to light irradiated in the large intestines, and blood vessel patterns.

The processor 140 may display the first visual effect of representing the wrinkle in each of the section images (S602).

The first visual effect may include a visual effect displayed by each of the markers on the corresponding wrinkle in each of the section images, and the size of each of the markers may be determined based on the size of the corresponding wrinkle.

The processor 140 may determine whether the rear surface of the wrinkle has been photographed for each of the section images (S603).

The processor 140 may display a second visual effect of representing, in at least one of the section images, in which a rear surface of a wrinkle has not been photographed, that a rear surface of a wrinkle in the at least one of the section images has not been photographed (S604).

The processor 140 may delete the second visual effect when the rear surface of the corresponding wrinkle is photographed (S605).

The processor 140 may delete the second visual effect displayed in the corresponding wrinkle to allow the medical expert to easily recognize that the rear surface of the corresponding wrinkle has been completely photographed.

Although FIG. 6 describes that operations S601 to S605 are sequentially performed, this is merely an exemplary description of the technical spirit of the present embodiment, and FIG. 6 is not limited to a time-series sequence because the order of FIG. 6 may be changed or one or more of operations S601 to S605 are performed in parallel so that FIG. 6 may be variously corrected and modified to be applied by an ordinary person in the art, to which the inventive concept pertains, without departing from the intrinsic features of the present embodiment.

The method according to the inventive concept described above may be coupled to the server that is a hardware element to be realized in a program (or an application) to be executed and may be stored in a medium.

The program may include a code that is coded in a computer language, such as C, C++, JAVA, or a machine language, by which a processor (CPU) of the computer may be read through a device interface of the computer, to execute the methods implemented by a program after the computer reads the program. The code may include a functional code related to a function that defines necessary functions that execute the methods, and the functions may include an execution procedure related control code necessary to execute the functions in its procedures by the processor of the computer. Further, the code may further include additional information that is necessary to execute the functions by the processor of the computer or a memory reference related code on at which location (address) of an internal or external memory of the computer should be referenced by the media. Further, when the processor of the computer is required to perform communication with another computer or server in a remote site to allow the processor of the computer to execute the functions, the code may further include a communication related code on how the processor of the computer executes communication with another computer or server or which information or medium should be transmitted and received during communication by using a communication module of the computer.

The storage medium refers not to a medium, such as a register, a cash, or a memory, which stores data for a short time but to a medium that stores data semi-permanently and is read by a device. In detail, an example of the storage medium may include a ROM, a RAM, a CD-ROM, a magnetic tape, a floppy disk, and an optical data storage device, but the present invention is not limited thereto. That is, the program may be stored in various recording media on various servers, which the computer may access, or in various recording media on the computer of the user. Further, the media may be dispersed in a computer system connected through a network, and codes that may be read by the computer in a dispersion manner may be stored in a distributed manner

The operations of a method or an algorithm that have been described in relation to the embodiments of the inventive concept may be directly implemented by hardware, may be implemented by a software module executed by hardware, or may be implemented by a combination thereof. The software module may reside in a random access memory (RAM), a read only memory (ROM), an erasable programmable ROM (EPROM), an electrically erasable programmable ROM (EEPROM), a flash memory, a hard disk, a detachable disk, a CD-ROM, or a computer readable recording medium in an arbitrary form, which is well known in the art to which the inventive concept pertains.

The inventive concept may have an effect of easily discovering a polyps that may be located behind a wrinkle of a large intestine by recognizing the wrinkle of the large intestine in an endoscope image of the large intestine and providing the endoscope image of the large intestine to an expert when a large intestine endoscope is performed.

In detail, the inventive concept may have an effect of causing a wrinkle to be identified as a whole while only a portion of the wrinkle is identified when the size of the wrinkle is large, by displaying the large intestine recognized in an endoscope image of a large intestine such that visual effects are different according to the size of the wrinkle of the large intestine.

Furthermore, the inventive concept may have an effect of clearly determining a site, at which a rear surface of a wrinkle has been identified, and a site, at which a rear surface of a wrinkle has not been identified, by providing a visual effect for a corresponding wrinkle of a large intestine when the rear surface of the wrinkle has not been photographed through the endoscope, and removing a visual effect for the corresponding wrinkle when an expert has photographed the rear surface of the corresponding wrinkle by using the endoscope.

The effects of the inventive concept are not limited thereto, and other unmentioned effects of the inventive concept may be clearly appreciated by those skilled in the art from the following descriptions.

Although the exemplary embodiments of the inventive concept have been described with reference to the accompanying drawings, it will be understood by those skilled in the art to which the inventive concept pertains that the inventive concept can be carried out in other detailed forms without changing the technical spirits and essential features thereof. Therefore, the above-described embodiments are exemplary in all aspects, and should be construed not to be restrictive.

What is claimed is:

1. A method for guiding an inspection of a large intestine by using an endoscope, being performed by an apparatus, the method comprising:
receiving an image captured by the endoscope introduced into the large intestine of a patient, in real time;
recognizing each of section images containing at least one wrinkle in the large intestine, in the image;
displaying a first visual effect of representing the wrinkle in each of the section images;
determining whether a rear surface of the wrinkle in each of the section images is photographed; and
displaying a second visual effect of representing, in at least one of the section images, in which a rear surface of a wrinkle has not been photographed, that the rear surface of the wrinkle in the at least one of the section images has not been photographed.

2. The method of claim 1, wherein the recognition is performed based on light irradiated into the large intestine by the endoscope.

3. The method of claim 1, wherein the recognition includes recognizing each of the section images through a deep learning model.

4. The method of claim 3, wherein the deep learning model is a model that is machine-learned based on wrinkle data in images of the large intestines of a plurality of patients, which are obtained from external annotators, and change amounts of shades due to light irradiated in the large intestines, and blood vessels.

5. The method of claim 1, wherein the first visual effect includes a visual effect of displaying a marker on the corresponding wrinkle in each of the section images, and
wherein a size of the marker is determined based on a size of the corresponding wrinkle.

6. The method of claim 1, further comprising:
when the rear surface of the corresponding wrinkle is photographed, deleting the second visual effect.

7. The method of claim 1, wherein a section of each of section images is set to a section from a point, at which the endoscope is introduced, to a point that is distinguished according to light irradiated into the large intestine by a lighting provided in the endoscope.

8. An apparatus for providing guide for an inspection of a large intestine by using an endoscope, the apparatus comprising:
a communication unit configured to receive an image captured by the endoscope introduced into a large intestine of a patient, in real time; and
a processor configured to:
recognize each of section images containing at least one wrinkle in the large intestine, in the image;
display a first visual effect of representing the wrinkle in each of the section images;
determine whether a rear surface of the wrinkle in each of the section images is photographed; and
display a second visual effect of representing, in at least one of the section images, in which a rear surface of a wrinkle has not been photographed, that the rear surface of the wrinkle in the at least one of the section images has not been photographed.

9. The apparatus of claim 8, wherein the processor recognizes each of the section images based on light irradiated into the large intestine by the endoscope.

10. The apparatus of claim 8, wherein the processor recognizes each of the section images through a deep learning model.

11. The apparatus of claim 10, wherein the deep learning model is a model that is machine-learned based on wrinkle data in images of the large intestines of a plurality of patients, which are obtained from external annotators, and change amounts of shades due to light irradiated in the large intestines, and blood vessels.

12. The apparatus of claim 11, wherein the processor generates the change amounts of the shades by determining whether the shades generated according to a light irradiated from a lighting of the endoscope to the wrinkle are changed by a preset threshold value or more.

13. The apparatus of claim 12, wherein the processor determines that there is a wrinkle when the shade is changed to have a darkness of a preset threshold value or more.

14. The apparatus of claim 11, wherein the processor recognizes a blood vessel pattern made by the blood vessel in the image of the large intestine, and recognizes an area in the image, in which the wrinkle is present, based on the recognized blood vessel pattern.

15. A program stored in a computer readable recording medium to execute the method of claim 1 through coupling to a computer.
